(19) United States
(12) Patent Application Publication
(10) Pub. No.: US 2022/0192574 A1
(43) Pub. Date: Jun. 23, 2022

(54) Implantable Medical Device Having a Processor Device for Detecting Cardiac Activity

(71) Applicant: BIOTRONIK SE & Co. KG, Berlin (DE)

(72) Inventors: Swetha VENNELAGANTI, King City, OR (US); Garth GARNER, Tigard, OR (US); R. Hollis WHITTINGTON, Portland, OR (US)

(73) Assignee: BIOTRONIK SE & Co. KG, Berlin (DE)

(21) Appl. No.: 17/599,884

(22) PCT Filed: Jan. 27, 2020
(86) PCT No.: PCT/EP2020/051940

§ 371 Date: Sep. 29, 2021

Related U.S. Application Data
(60) Provisional application No. 62/844,765, filed on May 8, 2019.

(51) Int. Cl. A61B 5/352; A61B 5/283; G16H 20/40; G16H 40/67

(52) U.S. Cl. CPC A61B 5/352; G16H 40/67; G16H 20/40; A61B 5/283

(57) ABSTRACT

An implantable medical device comprises a sensor device for obtaining a signal indicative of cardiac activity within a patient, and a processor device configured to process said signal obtained using the sensor device. The processor device is configured to detect a peak indicative of a cardiac event in said signal by comparing said signal to a sense threshold. The processor device in addition is configured to adaptively control said sense threshold such that the sense threshold in at least one time period assumes a value which is constant over said at least one time period, wherein the sense threshold is reduced after lapse of said at least one time period.

### CROSS-REFERENCE TO RELATED APPLICATIONS

This application is the United States national phase under 35 U.S.C. § 371 of PCT International Patent Application No. PCT/EP2020/051940, filed on Jan. 27, 2020, which claims the benefit of U.S. Patent Application No. 62/844,765, filed on May 8, 2019, the disclosures of which are hereby incorporated by reference herein in their entireties.

### TECHNICAL FIELD

The present invention relates to an implantable medical device according to the preamble of claim 1 and to a method for operating an implantable medical device.

### BACKGROUND

An implantable medical device of this kind comprises a sensor device for obtaining a signal indicative of cardiac activity within a patient. The implantable medical device zo furthermore comprises a processor device which is configured to process said signal obtained using the sensor device in that the processor device detects a peak indicative of a cardiac event in the signal by comparing said signal to a sense threshold.

An implantable medical device of this kind may for example be a monitoring device which is implanted in a patient such that cardiac signals, in particular in the shape of an electrocardiogram, may be detected in order to monitor cardiac activity. Such monitoring device may for example be subcutaneously implanted in a patient and hence is placed not within the heart, but subcutaneously in the vicinity outside of the heart. A monitoring device of this kind may be suited to remain within a patient over a prolonged period of time, for example several months or even years, such that a continuous monitoring of a patient's cardiac health may be achieved.

A monitoring device may for example be configured as a so-called loop recorder which repeatedly takes measurements (so-called snapshots) in an implanted state and, for recording such measurements, overwrites previous measurements. Such monitoring device shall be energy-efficient and may be configured for example to transmit recorded data to an external device outside of a patient by employing a suitable communication technology.

For an energy efficient operation, herein, the monitoring device beneficially does not record data continuously, but records only snapshots of cardiac activity which are indicative of an abnormal behavior, for example a bradycardia.

For detecting cardiac activity, typically a signal is processed to derive an electrocardiogram, which comprises waveforms indicative of cardiac events, such as a so-called QRS waveform and a T wave. By monitoring a series of QRS waveforms, conclusions with respect to the heart rate and potential arrhythmias can be drawn.

One issue in this respect is that waveforms in an electrocardiogram may have a variable amplitude. For example, a QRS waveform having peaks of large amplitude may be followed by a QRS complex being formed by peaks of a substantially smaller amplitude. In addition, ectopies) may occur, which fall outside of the rhythmic pattern of regular QRS waveforms and relate to an electrical irritability in the myocardium. An undersensing of cardiac events, namely QRS waveforms or ectopy may cause a recording of false cardiac activity snapshots and a false detection of bradycardia and asystole, which shall be avoided.

U.S. Publication No. 2013/0237868 A1 discloses a method of controlling a threshold for detecting peaks of physiological signals. In the method a physiological signal measured from a person is obtained, and it is determined whether a peak of the physiological signal is detected based on a result of comparing the physiological signal with a threshold. The threshold herein may be controlled such that it continuously reduces towards a minimum value.

The present disclosure is directed toward overcoming one or more of the above-mentioned problems, though not necessarily limited to embodiments that do.

### SUMMARY

It is an object of the present invention to provide an implantable medical device and a method for operating an implantable medical device which allow for a reliable detection of cardiac events even in case of absolutely or relatively small signal amplitudes related to cardiac events or in the case of absolutely or relatively large signal amplitudes related to cardiac events. For instance, cardiac ectopies as Premature Ventricular Contractions (PVCs), can have a larger or smaller amplitude compared to the surrounding elevations of an ECG, based on the direction of the conduction of the event. According to embodiments of the present invention, the proposed solutions address both cases.

At least this object is achieved by means of an implantable medical device comprising the features of claim 1.

Accordingly, the processor device is configured to adaptively control said sense threshold such that the sense threshold in at least one time period assumes a value which is constant over said at least one time period, wherein the sense threshold is reduced after lapse of said at least one time period.

The processor device is configured to detect a peak in a signal, in particular an electrocardiogram signal, as obtained from a sensor device by observing whether the signal crosses a sense threshold. If the signal crosses said sense threshold, this is indicative of a cardiac event, for example a QRS waveform in the signal or a so-called cardiac ectopy, which shall be detected and potentially recorded.

Herein, in order to avoid an undersensing of a cardiac event, the sense threshold beneficially is reduced with respect to an initial starting value of the sense threshold, such that the sense threshold is adapted to allow for a detection of low amplitude cardiac events. However, following a previous peak, for at least one time period the sense threshold is kept constant. For example, for some time following a detected peak the sense threshold may be kept constant at a rather high value, in order to then reduce the sense threshold following that time period. This makes sure that in a time period following a previous peak it is unlikely that another peak within a short time range following a peak is detected, in that a peak is only detected if it crosses the sense threshold, the sense threshold being higher close to the previous peak then at some temporal distance with respect to the previous peak.

Generally, different scenarios may exist which may cause an undersensing of events and hence a false detection of bradycardia and asystole. For example, an undersensing may occur after a cardiac ectopy of large amplitude has occurred. In another example, an undersensing of ectopies having a small amplitude may occur. In yet another example an undersensing of ectopies in a close time range with respect to a previous peak associated with a prior cardiac event may occur. Such undersensing events generally shall be avoided in order to provide for a reliable detection of cardiac events, even if cardiac events are associated only with small amplitude waveforms in a cardiac signal, in absolute terms or relative to a prior waveform.

In one embodiment, the processor device is configured to identify a maximum peak value within a peak detection window subsequent to a crossing of the sense threshold by said signal. If the signal crosses, at a specific point in time, the sense threshold, a cardiac event is identified and a peak detection window is started. Within the peak detection window the signal is tracked and a maximum amplitude of the signal within the peak detection window is stored in a register as the maximum peak value.

In one embodiment, based on the maximum peak value, then, a starting value for the sense threshold for a detection of a subsequent peak may be set. Namely, the starting value of the sense threshold in one embodiment is set making use of a reference threshold which is derived from the maximum peak value of the prior peak. For example, the threshold reference may be set, at least in an initial time period, to correspond to the maximum peak value. Alternatively, the reference threshold may be set to a value below the maximum peak value, for example to a value corresponding to a certain percentage of the maximum peak value. Different settings herein may employ different starting values, which in different ways relate to the maximum peak value of a peak detected in a prior peak detection window.

In order to avoid that the starting value of the sense threshold is set to an (excessively) large value in case of a large amplitude cardiac event, for example a cardiac ectopy exhibiting a large amplitude, it may be desirable to limit the starting value. For this, in one embodiment, the processor device may be configured to set the reference threshold to a value dependent on the maximum peak value if and only if this value does not exceed a predefined reference absolute threshold. If the value dependent on the maximum peak value exceeds the reference absolute threshold, the reference threshold instead may be set to the reference absolute threshold, such that the reference threshold is chosen to correspond to the minimum of the value dependent on the maximum peak value and the reference absolute pressure.

According to an embodiment, the processor is configured to set the reference threshold to a minimum threshold in case no cardiac activity has been sensed for a predetermined period of time. That minimum threshold can be an absolute minimum threshold.

The reference absolute threshold may for example lie in a range between 0 and 2 mV, for example in between 0.2 mV and 1 mV, for example between 0.3 mV and 0.4 mV, for an electrocardiogram signal picked up by the implantable medical device.

In one embodiment, the reference absolute threshold is fixed and is not changed during operation of the implantable medical device. In this case, the reference absolute threshold may for example be fixedly programmed within the processor device.

In an alternative embodiment, the reference absolute threshold may itself be adaptive in that it is determined based on a number of prior peaks, for example at least two previous peaks. For example, the reference absolute threshold may be set according to the average of maximum peak values of a defined number of previous peaks, for example two or more previous peaks. In this way, individual variations in signal amplitude for any patient population may be taken into account.

For example, the reference absolute threshold may be set to correspond to a predefined percentage of the average of maximum peak values of the defined number of previous peaks.

In one embodiment, the processor device is configured to start detection of a subsequent peak once a detection hold-off period has elapsed after a crossing of the sense threshold and hence after detection of a previous peak. The detection hold-off period prevents that after a detection of a peak immediately another peak is detected. Rather, following the detection of a peak (in the event of a crossing of the sense threshold) the detection hold-off period is started, in which no further detection of a subsequent peak is possible. The detection hold-off period hence resembles a blanking window in which no peak detection is possible.

In one embodiment, the processing device is configured to control the sense threshold such that the sense threshold is kept constant for a predefined time period following the detection hold-off period. After the detection hold-off period has elapsed, a peak again may be detected, wherein for this the sense threshold is suitably set.

Herein, the sense threshold in one embodiment starts at a starting value and is kept constant for a predefined time period at the starting value, before it is reduced in order to approach towards a target threshold. Hence, immediately following the detection hold-off period the sense threshold may be kept at a rather high threshold value and may be reduced only subsequently in order to allow for a detection of a subsequent peak.

In one embodiment, the processor device is configured to control said sense threshold such that the sense threshold is kept constant in a delay time period immediately following the detection hold-off period. In the delay time period the sense threshold may be set to an increased value, such that with within the delay time period the likelihood for detection of another cardiac event is reduced. The delay time period herein may have a fixed time width or may be adaptive, for example in that the width of the delay time period is changed based on the maximum amplitude value of a prior detected peak.

For example, if a prior peak has a large maximum amplitude value, the delay time period may be set to a small value, such that the sense threshold is reduced in a faster manner to approach towards a reduced target threshold. If, in turn, the prior peak exhibits a small amplitude, a longer delay time period may be used, such that the sense threshold is kept at a higher value for a longer time when encountering small signal amplitudes. This delays the start of a sense threshold countdown, thereby resulting in a slower countdown for small signals, which may help for a better sensing for smaller signals, which otherwise may be prone to oversensing due to noise.

In the context of the present invention, oversensing is understood as erroneously detecting activities in an ECG using a detection algorithm. Oversensing takes place e.g. if a sensing threshold for detecting a certain type of cardiac event is set too low, so that other cardiac activities in the ECG are identified as the cardiac event of interest as well. On the other is hand, undersensing is understood as missed detections of (a) cardiac event(s) in an ECG. Undersensing may occur e.g. if a sensing threshold is set too high, so the cardiac event of interest, having a lower amplitude than the sensing threshold would be overseen by the algorithm.

In one embodiment, the processor device is configured to set the delay time period to a first value if the detected peak has a peak value (maximum amplitude value) above a low signal threshold, and to a second value if the detected peak has a peak value below said low signal threshold. Hence, if the prior detected peak lies above the low signal threshold, the delay time period may be set to a first value, which may be rather short. If, in turn, the peak value of the prior peak lies below the low signal threshold, the delay time period is set to a second value, which may be larger than the first value such that a countdown for the sense threshold to reduce the sense threshold towards a target threshold is delayed.

In one embodiment, the processor device is configured to control the sense threshold such that the sense threshold is reduced in steps for a series of multiple time periods until a predefined target threshold is reached. Hence, the sense threshold is reduced in a stepwise manner, wherein some or all of the time periods may have an equal time length and hence the sense threshold may be reduced in steps having a constant width. The reduction takes place until a predefined target threshold is reached, such that the sense threshold may not drop below the target threshold, but assumes the value of the target threshold once the target threshold is reached.

The length of the time period may be set according to user-defined settings. For example, the time length may assume a value in between 50 ms and 500 ms, for example in between 100 ms and 300 ms, wherein in different settings different values may be chosen, a smaller value for the time length causing a faster rate of reduction towards the target threshold.

The stepwise reduction may take place by reducing the sense threshold by a certain margin once the end of a time period is reached. Herein, the sense threshold may be set in a subsequent time period as a percentage value of the sense threshold in a previous time period.

For example, in a default setting the sense threshold in a time period may be set to a value in between 50% and 95%, for example between 60% and 90%, of the sense threshold in the previous time window. Herein, dependent on the specific setting, the percentage may be adapted. In one example, in one setting a default rate of reduction may be defined by a percentage value of 75%. If the rate of reduction shall be slowed down, the percentage may for example be set to 87.5%. If the rate of reduction shall be increased, the percentage may be set to 62.5%, wherein the rate of reduction may be defined and chosen for example by a user defined setting.

By suitably choosing the setting, an undersensing of cardiac events may be prevented. For example, an undersensing after a large prior cardiac event, for example a large ectopy, may be avoided for example by choosing a lower starting value for the sense threshold.

An undersensing of small cardiac events, for example small premature current ventricular contractions, and/or an undersensing of a cardiac event following within a close temporal distance to a prior cardiac event, may be avoided for example by choosing a faster reduction by suitably setting the time length of the time windows and/or by increasing the percentage for reducing the sense threshold.

At least the object is also achieved by a method for operating an implantable medical device, comprising: processing, using a processor device of the implantable medical device, a signal indicative of cardiac activity within a patient and obtained by a sensor device for detecting a peak indicative of a cardiac event in said signal by comparing said signal to a sense threshold; and adaptively controlling said sense threshold such that the sense threshold in at least one time period assumes a value which is constant over said time period, wherein the sense threshold is reduced following said at least one time period.

The advantages and advantageous embodiments described above for the implantable medical device equally apply also to the method, such that in this respect it shall be referred to the above.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

One or more objects underlying the present invention shall subsequently be explained in more detail with reference to the embodiments shown in the drawings. Herein:

FIG. 1 shows a schematic drawing of a medical device in the shape of a monitoring device in an implanted state in a patient;

FIG. 2 shows a schematic drawing of an implantable medical device in the shape of a monitoring device;

FIGS. 3A-3E show different electrocardiogram signals exhibiting different waveforms relating to cardiac events;

FIG. 4 shows an illustration of a sensing of a cardiac event in an electrocardiogram signal using a sense threshold;

FIG. 5 shows the application of a reference absolute threshold for setting a starting value of a sense threshold;

FIG. 6 shows the adaption of a time window based on a peak value of a detected peak;

FIG. 7 shows sense thresholds for different settings;

FIG. 8 shows sense thresholds for another example of different settings; and

FIG. 9 shows sense thresholds for yet another example of different settings.

### DETAILED DESCRIPTION

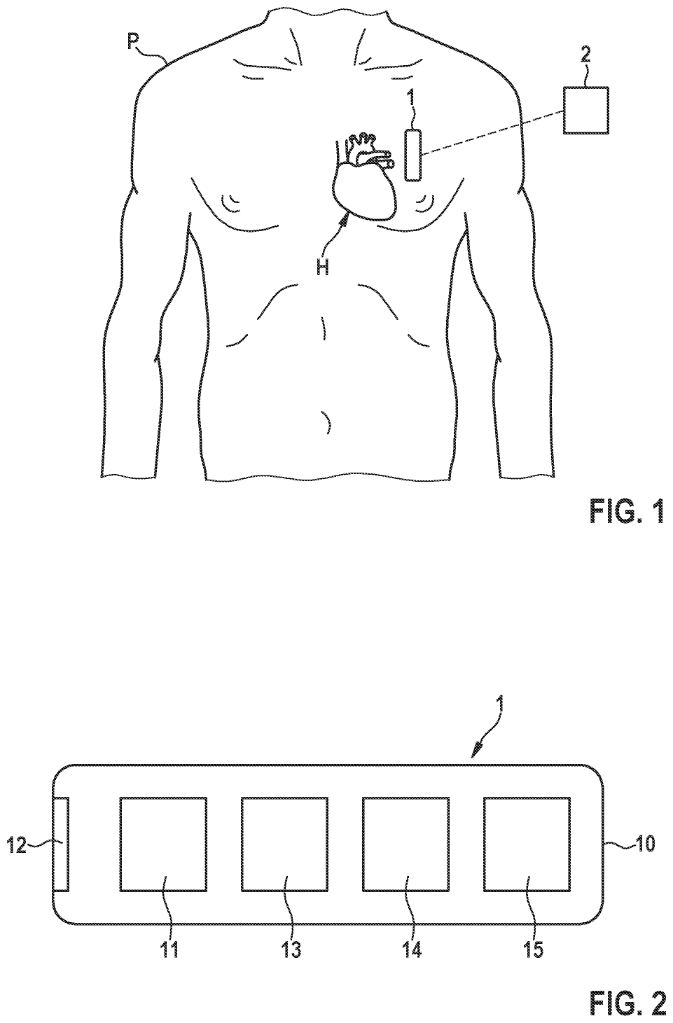

FIG. 1 shows an implantable medical device 1 in an implanted state within a patient P. The implantable medical device 1 functions as a monitoring device and is implanted close to the heart H of the patient P, the implantable medical device 1 being enabled to communicate with an external device 2 to transfer measurement data to the external device 2.

The implantable medical device 1 for example may have the shape of a loop recorder which is configured to record data, wherein actual data may overwrite previous data in a looping fashion.

A medical device 1 in the shape of a monitoring device shall remain within a patient P over a prolonged period of time, for example several months or even years. For this, the medical device 1 shall operate in an energy-efficient manner, in that data shall be recorded and transferred to an external device 2 only if an abnormal behavior, for example a bradycardia or an asystole, is detected. A false recording of data, in the shape of measurement snippets also denoted as snapshots, herein shall be avoided.

Referring now to FIG. 2, an implantable medical device 1 comprises a processor device 11 cooperating with a sensor device 12 for sensing a sensing signal relating to activity of a patient's heart H. The sensor device 12 may for example comprise an electrode for electrically sensing electrical signals originating from the heart H and in particular corresponding to ventricular contractions of the heart H, such that by means of the medical device 1 a signal in the shape of an electrocardiogram may be recorded.

The implantable medical device 1 in addition comprises a memory device 13 serving to store recorded data, an energy storage 14 in the shape of a battery and a communication device 15 in the shape of circuitry for establishing a communication connection to an external device 2 for transferring recorded data (snapshots) to the external device 2 and for receiving e.g. control commands or programming data, for example relating to certain settings of the medical device 1, from the external device 2.

The medical device 1 comprises a housing 10 which encapsulates the components received within in a fluid-tight manner.

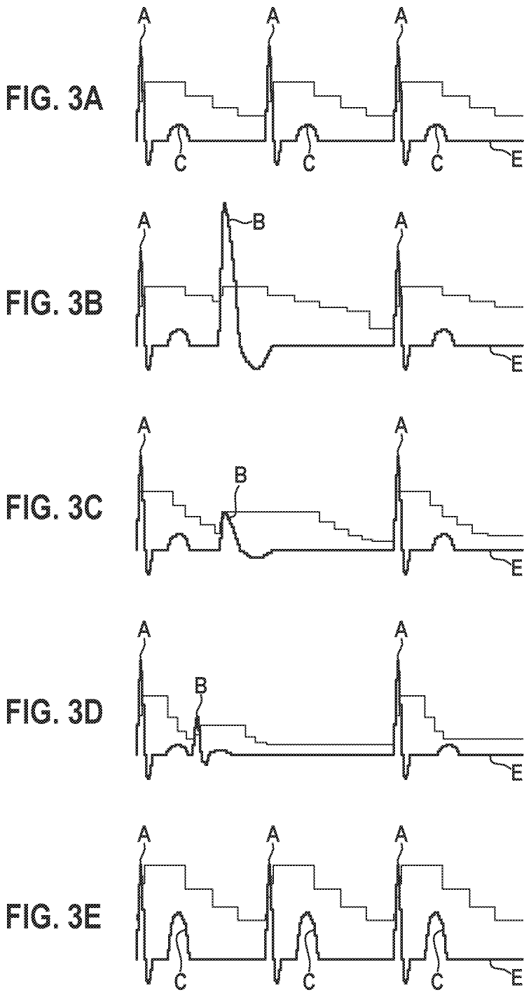

Generally, referring now to FIGS. 3A to 3E, in an electrocardiogram E cardiac activity can be identified according to specific waveforms, namely so-called QRS waveforms A which are regularly followed by so-called T waves C in a periodic fashion. A QRS waveform A herein comprises peaks of fairly large amplitude, the peaks of a QRS waveform A usually far exceeding a subsequent T wave C. Generally, according to the sequence of QRS waveforms A the heart rate can be determined, as it is visible from FIG. 3A.

Even in a healthy heart it is not uncommon that ectopies B (in short PVC) may occur singly or in repeated patterns, such ectopies B being caused by electrical irritability in the ventricular conduction system. Such ectopies B interrupt the regular pattern of QRS waveforms A and may have a signal amplitude substantially larger than a QRS waveform A, as shown in FIG. 3B, or substantially smaller than a QRS waveform A, as shown in FIG. 3C. In addition, such ectopies B may occur at a substantial temporal distance with respect to a prior QRS waveform A, as it is the case in FIGS. 3B and 3C, or may occur within a rather close distance to a prior QRS waveform A, as it is the case in FIG. 3D.

Furthermore, T waves C may have a small signal amplitude, as in FIGS. 3A to 3D, but may also have a fairly large amplitude, as it is visible in FIG. 3E.

There generally is a desire to be able to detect the occurrence of QRS waveforms A as well as ectopies B. At the same time, QRS waveforms A and ectopies B shall be distinguished from T waves C, which shall not be falsely detected and identified as a QRS peak or an ectopy.

Generally, according to detected QRS waveforms A and ectopies B the heart rate shall be determined, and, if an abnormal pattern from the heart rate is detected, a snapshot shall be is recorded and potentially transferred to an external device 2. Herein, if peaks relating to a QRS waveform A or a ectopy B are missed, this may lead to a false reading of the heart rate and hence to an erroneously taken snapshot, and a potentially erroneous detection of a bradycardia and asystole.

A primary cause of such false snapshots and false identification of bradycardia and asystole is an undersensing caused by ectopies B. Herein, three types of undersensing events may generally occur, namely an undersensing after a large ectopy B, an undersensing of small ectopies B, and an undersensing of a ectopies B following within a close time range to a prior QRS waveform A.

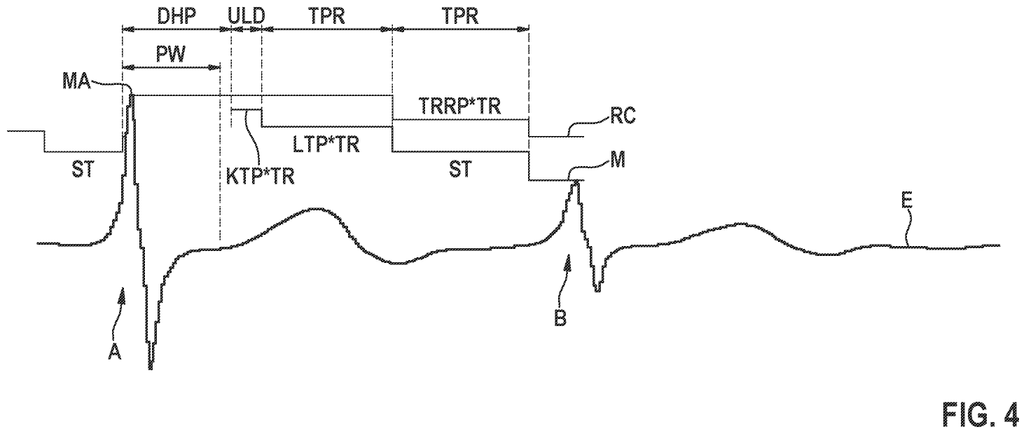

Referring now to FIG. 4, within the instant text a scheme is proposed which allows for a reliable detection of peaks relating to QRS waveforms A and ectopies B.

The detection of a peak relating to a QRS waveform A or a ectopy B generally takes place by using a sense threshold ST, wherein a signal corresponding to an electrocardiogram E as picked up by a sensor device 12 (see FIG. 2) is compared to the sense threshold ST, and if the signal crosses the sense threshold ST it is found for a peak relating to a QRS waveform A or a ectopy B (wherein it is not necessarily distinguished between a QRS waveform A or a ectopy B, but merely the rhythmic pattern and from this the heart rate is determined).

It herein is proposed to use a time-variable sense threshold ST, which successively is reduced towards a target threshold M, wherein the manner and pattern of reducing the sense threshold ST may be adaptive to be able to detect peaks of small amplitude, for example relating to ectopies B, as indicated in FIG. 4.

In the scheme of FIG. 4, a peak is assumed to be detected if the signal E crosses the sense threshold ST, upon which a peak detection window PW is started and, within the peak detection window PW, the signal amplitude is tracked in a threshold reference register. In this way a maximum peak value MA within the peak detection window PW is determined and stored, such that it can be used to set the sense threshold ST in a subsequent detection is phase.

Also, upon the crossing of the sense threshold ST by the signal E, a detection hold-off period DHP is started, in which no detection of a peak shall take place, such that no additional peak can be detected within a certain distance to a prior peak.

The detection hold-off period DHP may be equal in length to the pulse detection window PW, but may, as visible from FIG. 4, also differ in length to the pulse detection window PW.

Following the detection hold-off period DHP, a new sense threshold ST is set, wherein the sense threshold ST is derived from the peak measurement within the pulse detection window PW by making use of the maximum peak value MA as determined in the peak detection window PW. In particular, a starting value of the sense threshold ST may be set as a certain percentage of the maximum peak value MA as determined in the pulse detection window PW.

Herein, as visible from FIG. 4, in one embodiment at the beginning of the new detection phase the sense threshold ST is set according to an upper threshold UTP within a delay time period ULD (also denoted as upper-to-lower delay). Within the delay time period ULD the sense threshold ST is set to a value equal to UTP times TR, wherein TR is a threshold reference equal to the maximum peak value MA and UTP corresponds to a percentage value, for example in a range between 80 to 95%.

Upon expiration of the delay time period ULD, the sense threshold ST is set to a reduced value corresponding to a lower threshold LTP times the threshold reference TR, wherein LTP again is a percentage value, but being smaller than the percentage value UTP in the delay time period ULD. LTP for example may lie in a range between 60 to 90%.

The sense threshold ST within the delay time period ULD and in the time period TPR following the delay time period ULD is kept constant. Upon expiration of the time period is TPR (also denoted as threshold percentage reduction time) the sense threshold ST is reduced to a value TRRP times this threshold reference TR, wherein the reduction value TRRP corresponds to a percentage value by which the reference curve RC is reduced and, hence, also the sense threshold ST is reduced, as shown in FIG. 4.

After expiration of another time period TPR, the sense threshold ST again is reduced by a step, wherein the step again corresponds to a reduction by the percentage factor TRRP, in that the reference curve RC is reduced by multiplying the prior value of the reference curve RC by TRRP.

If a target threshold M—corresponding to a minimum value for the threshold below which the sense threshold ST shall not fall—is reached, the sense threshold ST assumes the value of the target threshold M and remains at the target threshold M.

If a subsequent peak, in the example of FIG. 4 an ectopy B, causes a crossing of the sense threshold ST, a peak again is detected, and the procedure starts anew. Herein, again, a sense threshold ST in a subsequent detection phase is set according to a now determined maximum peak value MA, such that the sense threshold ST in different detection phases may differ.

By using the scheme of FIG. 4, the sense threshold ST is reduced in steps, wherein the sense threshold ST is caused to be stepwise reduced towards the target threshold M. By suitably choosing the length of the time period TPR and the percentage values for the reduction, herein, the detection algorithm may be adapted for the sensing of small amplitude signals, wherein settings may be changed for different patients having different conditions and hence having a different likelihood of occurrence of different cardiac activity patterns.

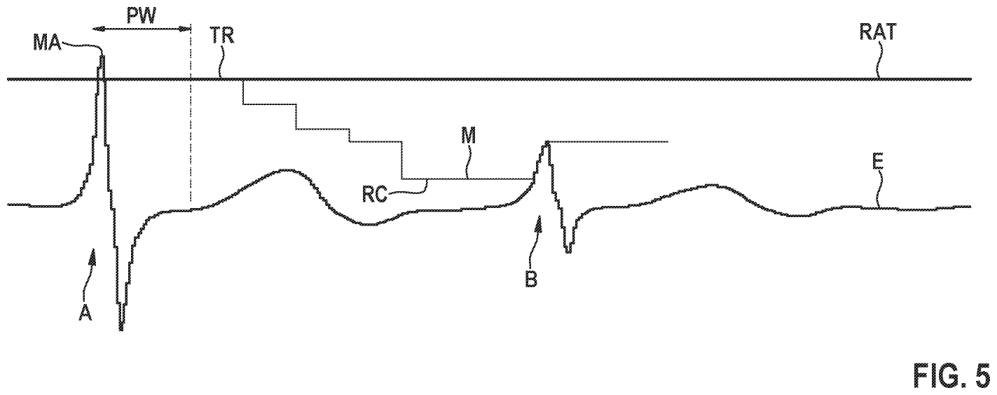

Referring now to FIG. 5, in one embodiment a reference absolute threshold RAT may be employed for setting the sense threshold ST in a detection phase. The reference absolute threshold RAT provides an upper limit for the reference threshold TR and hence for the reference curve RC, such that the threshold reference TR may not be set to a value exceeding the reference absolute threshold RAT. In particular, if the maximum peak value MA in a peak detection window PW for a detected peak is above the reference absolute threshold RAT, as visible from FIG. 5, the threshold reference TR is set to the reference absolute threshold RAT, and hence to a value smaller than the maximum peak value MA. If, however, the maximum peak value MA lies below the reference absolute threshold RAT, the threshold reference TR (which provides for the initial value of the reference curve RC) is set to the maximum peak value MA. Hence, the threshold reference TR initially is set to the minimum of the maximum peak value MA and the reference absolute threshold RAT.

The reference absolute threshold RAT may for example lie in a range between 0 and 2 mV for an electrocardiogram signal E.

The reference absolute threshold RAT herein may be fixedly programmed and hence may be constant throughout a lifetime of a medical device 1 in the shape of a monitoring device.

In an alternative embodiment, the reference absolute threshold RAT may in itself be adaptive in that its value may be set dynamically for example depending on a number (more than 1) of prior peak amplitudes, corresponding to the maximum peak values MA of a predefined number of previous peaks. For example, the reference absolute threshold RAT may be set as a certain percentage of the average of the number of predefined peak values, hence taking into account individual variations in signal amplitude for any patient population.

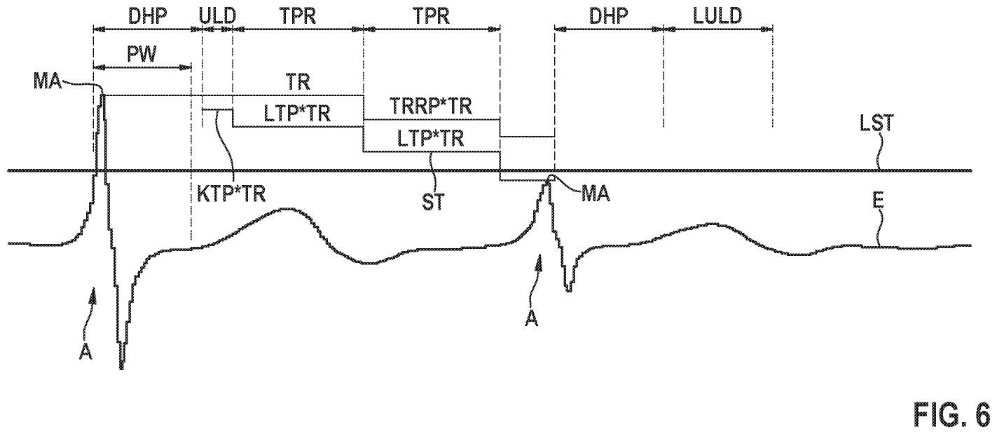

Referring now to FIG. 6, the length of the delay time period ULD may be adaptive. The delay time period ULD serves to avoid an oversensing of T waves C following a prior QRS waveform A, such that within the delay time period ULD an increased value for the sense threshold ST is set. Herein, when in the electrocardiogram signal E small amplitude QRS waveforms A and large amplitude QRS waveforms A are interspersed, it is advantageous to have a slower countdown for signals with small amplitude so that noise is not oversensed.

This may be achieved by using an amplitude threshold to determine a fast or slow countdown for each detected peak. If a maximum peak value MA lies above a low signal threshold LST, a regular delay time period ULD is used. If a maximum peak value MA instead lies below the low signals threshold LST, as in FIG. 6 for the QRS waveform A on the right, a long delay time period LULD is used, such that the countdown of the sense threshold ST towards a target threshold is delayed. Hence, a slower countdown for smaller signals is obtained, which may help for improving a sensing of small signals, which may be prone to oversensing due to noise.

Settings of the detection algorithm may be adapted to improve the detection of events of certain kinds and in certain scenarios.

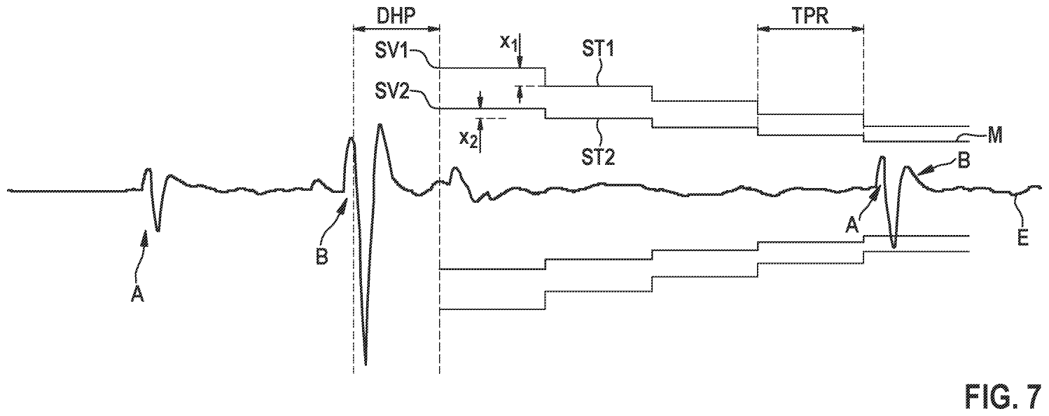

Referring now to FIG. 7, in a setting which may particularly be suited to provide for a reliable sensing after the occurrence of a large amplitude ectopy B, a starting value SV2 for the sense threshold ST2 may be reduced in comparison to a starting value SV1 of the sense threshold ST1 in a default setting, ST1 denoting the default sense threshold curve and ST2 denoting the sense threshold curve according to the adapted setting. By reducing the starting value SV2 (which may be achieved by adapting the percentage according to which the starting value SV2 is set) or by reducing the reference absolute threshold RAT (see FIG. 5), the sense threshold ST2 starts at a lower value and hence reduces faster towards the target threshold M, allowing for a detection of a subsequent peak relating to a low amplitude QRS waveform A following a large amplitude ectopy B, as visible from FIG. 7.

The adapted starting value SV2 may for example have a value in the range between 0.6 and 1 mV.

By reducing the starting value SV2, a countdown towards the target threshold M may be accelerated, allowing for example for a countdown towards the target threshold M within 1 second, in comparison to 2 seconds for a default setting.

Herein, furthermore, the rate of reduction may be adapted. Wherein for the default setting rather large steps X1 (relating to a reduction by repeatedly applying a percentage reduction, the sense threshold value in a time period being set to a certain percentage of the sense threshold value in the previous time period) for reducing the sense threshold ST1 may be employed, the step size X2 in the adapted setting may be reduced. For example, in the default setting (sense threshold ST1) the sense threshold value in a time period may be set to a value of 75% of the sense threshold value in a previous time period, wherein in the adapted setting (sense threshold ST2) the sense threshold value in a time period may be set to 87.5% of the sense threshold value in a previous time period. This slowing-down in the countdown towards the target threshold M for the adapted setting helps to prevent an oversensing of noise.

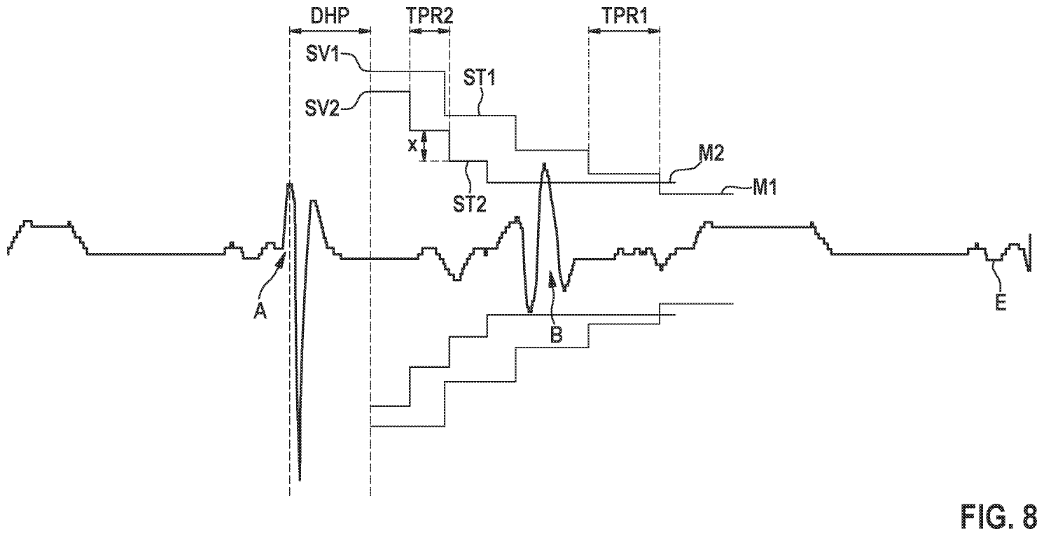

Referring now to FIG. 8, in a setting specifically adapted to allow for a sensing of small amplitude ectopies B, a smaller starting value SV2 for the sense threshold ST2 may be used in comparison to a default setting using a starting value SV1 for a sense threshold ST1. In addition, while using the same step size X for both settings, the length of the time period after which a reduction occurs may be shortened for the adapted setting, the adapted setting (sense threshold ST2) using a time period of length TPR2, in comparison to a length TPR1 of the default setting (sense threshold ST1).

For example, the starting value SV2 may be reduced to a value between 0.3 mV and 0.6 mV.

In addition, whereas the default setting may use a length TPR1 for each time period between 200 and 250 ms, in the adapted setting the length of the time period TPR2 may be reduced to a value between 100 and 150 ms.

In this way, the countdown towards the target threshold M may be accelerated, such that the countdown may take place within 1 seconds or shorter, in comparison to 2 seconds for the default setting.

In the setting of FIG. 8, an oversensing of noise may be avoided by using a long delay time period LULD, as shown in FIG. 6, assuming for example a low signal threshold LST in a range between 0.2 mV and 0.5 mg, for example at 0.3 mV. The long delay time period LULD may for example be set to a value between 300 ms and 800 ms, for example 500 ms.

In addition, different target thresholds M1, M2 may be employed for the default setting (sense threshold ST1) and the adapted setting (sense threshold ST2).

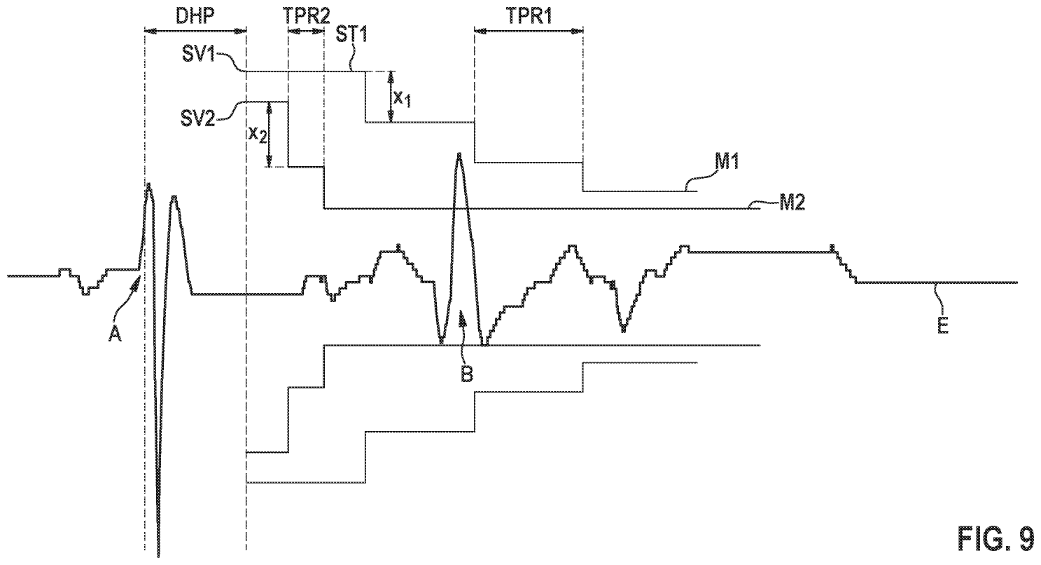

Referring now to FIG. 9, in order to be able to sense peaks within a short time range to a prior peak A, a faster countdown towards the target threshold may be achieved by limiting the start value SV2 and increasing the step size for the countdown. In the example of FIG. 9, the adapted setting uses a starting value SV2, which is smaller than the starting value ST1 for a default setting. In addition, a larger reduction step X2 in comparison to a step size X1 for a default setting is used. In particular, for the adapted setting a fast reduction of the sense threshold ST2 may be obtained by setting the sense threshold value in a time period to a rather small percentage of the sense threshold value in a previous time period, for example to a value in between 60 to 70% of the previous value, for example 62.5% in comparison to a default 75%.

Hence, a fast countdown is obtained, wherein the time period length TPR2 may for example be reduced to a value in between 50 ms and 100 ms, instead of a default time period length TPR1 in the range between 200 and 250 ms.

Also in the setting of FIG. 9, an oversensing of noise may be avoided by using a long delay time period LULD, as shown in FIG. 6, assuming for example a low signal threshold LST in a range between 0.2 mV and 0.5 mg, for example at 0.3 mV. The long delay time period LULD may for example be set to a value between 100 ms and 300 ms, for example 150 MS.

The idea of the present invention is not limited to the embodiments described above, but is may also be implemented in a different fashion.

Different settings for different scenarios may be employed, wherein the setting may be automatically adapted within the medical device, or may be adapted by a user to adapt the operation of the medical device to a certain patient exhibiting a certain state of cardiac health.

By means of the proposed scheme, a reliable detection of peaks following large amplitude ectopies as well as a reliable detection of small ectopies and ectopies within a short time range after a prior QRS waveform becomes possible. In this way, a false detection of bradycardia and asystole snapshots may be avoided, hence reducing a review burden for a physician.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
10 Housing
11 Processor device
12 Sensor device
13 Memory device
14 Energy storage
15 Communication device
2 External device
A QRS waveform
B Ectopy signal
C T wave
DHP Detection hold-off period
E ECG signal
LTP Lower Threshold
H Heart
LST Low signal threshold
LULD Long delay time period (long upper-to-lower delay)
M, M1, M2 Target threshold (minimum)
MA Maximum peak value
P Patient
PW Peak detection window
R Reference curve
RAT Reference absolute threshold
ST, ST1, ST2 Sense threshold
SV1, SV2 Start value
TPR Threshold percentage reduction time
TPR1, TPR2 Threshold percentage reduction time
TR Threshold reference
TRRP Threshold reference reduction percentage
ULD Delay time period (upper-to-lower delay)
UTP Upper threshold
X, X1, X2 Step size

1. An implantable medical device, comprising:
a sensor device for obtaining a signal indicative of cardiac activity within a patient; and
a processor device configured to process said signal obtained using the sensor device, wherein the processor device is configured to detect a peak indicative of a cardiac event in said signal by comparing said signal to a sense threshold,
wherein the processor device is configured to adaptively control said sense threshold such that the sense threshold in at least one time period assumes a value which is constant over said at least one time period, wherein the sense threshold is reduced after lapse of said at least one time period.

2. The implantable medical device of claim 1, wherein said processor device is configured to identify a maximum peak value within a peak detection window subsequent to a crossing of the sense threshold by said signal.

3. The implantable medical device of claim 2, wherein said processor device is configured to set a starting value of said sense threshold for detecting a subsequent peak based on a threshold reference derived from said maximum peak value.

4. The implantable medical device of claim 3, wherein said processor device is configured to set the threshold reference to a value dependent on said maximum peak value, or, if said value dependent on said maximum peak exceeds a reference absolute threshold, to the reference absolute threshold.

5. The implantable medical device of claim 4, wherein said reference absolute threshold is a fixed value, or is adaptively determined based on peak amplitude values of at least two previous peaks.

6. The implantable medical device of claim 1, wherein said processor device is configured to start detection for a subsequent peak once a detection hold-off period has elapsed after a crossing of the sense threshold by said signal.

7. The implantable medical device of claim 6, wherein said processor device is configured to control said sense threshold such that the sense threshold is kept constant for a predefined time period following said detection hold-off period.

8. The implantable medical device of claim 6, wherein said processor device is configured to control said sense threshold such that the sense threshold is kept constant in a delay time period immediately following said detection hold-off period.

9. The implantable medical device of claim 8, wherein said processor device is configured to adaptively set a time length of said delay time period based on a detected peak.

10. The implantable medical device of claim 9, wherein said processor device is configured to set the delay time period to a first value if the detected peak has a maximum peak value above a low signal threshold, and to a second value if the detected peak has a maximum peak value below said low signal threshold.

11. The implantable medical device of claim 10, wherein the second value is larger than the first value.

12. The implantable medical device of claim 1, wherein said processor device is configured to control said sense threshold such that the sense threshold is reduced in steps for a series of multiple time periods until a predefined target threshold is reached.

13. The implantable medical device of claim 12, wherein at least some of the time periods of said series of multiple time periods have an equal time length.

14. The implantable medical device of claim 1, wherein said processor device is configured to control said sense threshold such that the sense threshold in one time period is set as a percentage value of the sense threshold in a previous time period.

15. A method for operating an implantable medical device comprising:
processing, using a processor device of the implantable medical device, a signal indicative of cardiac activity within a patient and obtained by a sensor device for detecting a peak indicative of a cardiac event in said signal by comparing said signal to a sense threshold,
wherein said sense threshold is adaptively controlled such that the sense threshold in at least one time period assumes a value which is constant over said time period, wherein the sense threshold is reduced following said at least one time period.
